(19) United States
(12) Patent Application Publication
(10) Pub. No.: US 2022/0151963 A1
(43) Pub. Date: May 19, 2022

(54) PARENTERAL DELIVERY OF AVIZAFONE

(71) Applicant: Sollievo Pharmaceuticals, Inc, San Juan Capistrano, CA (US)

(72) Inventor: Robert Schultz, San Juan Capistrano, CA (US)

(73) Assignee: Sollievo Pharmaceuticals, Inc, San Juan Capistrano, CA (US)

(21) Appl. No.: 17/590,402

(22) Filed: Feb. 1, 2022

Related U.S. Application Data
(62) Division of application No. 17/334,671, filed on May 28, 2021, now abandoned.
(60) Provisional application No. 63/031,155, filed on May 28, 2020.

(51) Int. Cl. A61K 31/167; A61P 25/20; A61K 9/00

(52) U.S. Cl. CPC A61K 31/167; A61K 9/0019; A61P 25/20

(57) ABSTRACT

Water-stable formulations of Avizafone and methods of use are described herein. The formulations may be administered to patients intravenously, intramuscularly, or subcutaneously. For serious COVID, or other viral infections, Avizafone is a rapidly sedating benzodiazepine that could be used interchangeably with midazolam augmenting the supply of drugs for swift and intubation.

### CROSS-REFERENCE TO RELATED APPLICATIONS

This application is a divisional application of U.S. patent application Ser. No. 17/344,671, filed May 28, 2021, which claims the benefit of U.S. Provisional Application No. 63/031,155 filed May 28, 2020, which is incorporated herein by reference.

### BACKGROUND

### Field

The present disclosure relates generally to the field of pharmaceutical therapeutics, and more specifically to the administration of compounds capable of sedation, including compounds for the sedation of COVID patients.

### Description of the Related Art

Diazepam is a medication that is indicated for the management of anxiety disorders or for the short-term relief of the symptoms of anxiety. In a patient experiencing acute alcohol withdrawal, diazepam may be useful in the symptomatic relief of acute agitation, tremor, impending or acute delirium tremens, and hallucinations. Diazepam is also a useful adjunct for the relief of skeletal muscle spasm due to reflex spasm to local pathology, such as inflammation of the muscles or joints, or secondary to trauma. Diazepam is a useful adjunct in status epilepticus and severe recurrent convulsive seizures. It is also a useful premedication for relief of anxiety and tension in patients who are scheduled to undergo surgical procedures. The concentration of diazepam in the currently available commercial product is 0.0176 M.

One of the drawbacks of diazepam is that it is very poorly soluble in water. Per the package insert of the commercial drug product, to solubilize diazepam in a solution of sufficient concentration to be acceptable for a parenteral product, it is dissolved in a solution that is 40% propylene glycol, 10% ethyl alcohol, and 5% benzyl alcohol. When administered intravenously (IV), the commercial drug product requires a slow bolus (30 sec/mL) to prevent the drug from coming out of solution or causing significant irritation of the vein. It is recommended that a large vein should be used.

Other drawbacks of intramuscular (IM) or IV administration of commercial diazepam are: significant irritation and pain at the injection site, inability to add other pre-operation drugs to the same syringe due to risk of precipitation, inability to deliver the diazepam from an IV bag, inability to administer subcutaneously (SQ), and slow and erratic absorption of the drug upon IM injection due to the variability in fat content among patients.

In the late 1980s, the French military developed a water-soluble version of diazepam, avizafone (AVF), to use in a three-drug cocktail in combination with atropine and pralidoxime that was intended to be placed in an autoinjector for battlefield delivery of an antidote to nerve gas poisoning.

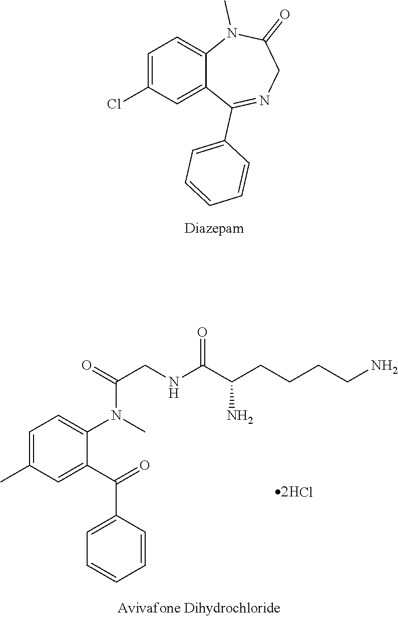 [SMILES: CN1C(=O)CN=C(c2ccccc2)c2cc(Cl)ccc21 ; Cc1ccc(N(C)C(=O)CNC(=O)[C@@H](N)CCCCN)c(C(=O)c2ccccc2)c1]

The Avizafone/Atropine/Pralidoxime combination was packaged in a unit dose syringe that was loaded into an autoinjector for use on the battlefield. To improve the stability of the Atropine, the solution in the military version was formulated at a pH of 4.0. A pH of 4.0 is more acidic than physiological pH of 7.4, and the lower pH increases the likelihood of inducing pain and inflammation when injected into muscle tissue.

During the height of COVID infections, hundreds of thousands of patients were put on life-saving ventilators to keep them breathing until the infection resolved. Midazolam, a benzodiazepine in the same class as Avizafone, is the drug of choice for sedating patients prior to intubation. Unfortunately, the unexpected demand for midazolam led to a shortage of this critical drug. Additionally, COVID infections induce metabolic imbalances and high fevers in many patients. Metabolic imbalances and high fevers can lead to severe agitation or delirium.

### SUMMARY

Some embodiments described herein relate to an injectable pharmaceutical formulation comprising an aqueous carrier and Avizafone or a pharmaceutically acceptable salt thereof, wherein the Avizafone is present in the aqueous carrier at a concentration of at least 0.05M.

In the event of a resurgence of serious COVID, or other viral infections, Avizafone is a rapidly sedating benzodiazepine that could be used interchangeably with midazolam augmenting the supply of drugs for intubation. When Avizafone is delivered intramuscularly by paramedics or hospital personnel, its swift sedation of the patient enables rapid diagnosis and acute treatment to begin.

### BRIEF DESCRIPTION OF THE DRAWINGS

FIG. 1 shows a graph of diazepam plasma levels in dogs over time after either IM or SQ administration of AVF.

FIG. 2 shows the time to onset of sedation in dogs after administration of AVF.

### DETAILED DESCRIPTION

Some embodiments described herein relate to an injectable pharmaceutical formulation comprising an aqueous carrier and Avizafone or a pharmaceutically acceptable salt thereof, wherein the Avizafone is present in the aqueous carrier at a concentration of at least 0.05M.

The term “pharmaceutically acceptable salt” includes salts of Avizafone in combination with an organic or inorganic acid or base. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, adipic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, benzenesulfonic acid, methanesulfonic acid, ethanesulfonic acid, p-toluenesulfonic acid monohydrate, (1S)-(+)-10-camphorsulfonic acid, (+)-7,7-dimethyl-2-oxobicyclo[2.2.1]heptane-1-methanesulfonic acid, 1,2-ethanedisulfonic acid, dodecyl sulfonic acid, salicylic acid, glucoheptonic acid, gluconic acid, glucuronic acid, hippuric acid, hydrochloride hemiethanolic acid, 2-hydroxyethanesulfonic acid, lactic acid, lactobionic acid, methylbromide acid, methyl sulfuric acid, 2-naphthalenesulfonic acid, oleic acid, 4,4′-methylenebis-[3-hydroxy-2-naphthalenecarboxylic acid], polygalacturonic acid, stearic acid, sulfosalicylic acid, tannic acid, terphthalic acid and the like. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts.

In some embodiments of the injectable pharmaceutical formulation, the Avizafone or a pharmaceutically acceptable salt thereof is present in the aqueous carrier at a concentration between 0.05M and 1.0M. In some embodiments, the Avizafone or a pharmaceutically acceptable salt thereof is present in the aqueous carrier at a concentration between 0.05M and 0.75M. In some embodiments, the Avizafone or a pharmaceutically acceptable salt thereof is present in the aqueous carrier at a concentration between 0.05M and 0.5M. In some embodiments, the Avizafone or a pharmaceutically acceptable salt thereof is present in the aqueous carrier at a concentration between 0.05M and 0.3M. In some embodiments, the Avizafone or a pharmaceutically acceptable salt thereof is present in the aqueous carrier at a concentration of at least 0.06M. In some embodiments, the Avizafone or a pharmaceutically acceptable salt thereof is present in the aqueous carrier at a concentration of at least 0.07M. In some embodiments, the Avizafone or a pharmaceutically acceptable salt thereof is present in the aqueous carrier at a concentration of at least 0.08M. In some embodiments, the Avizafone or a pharmaceutically acceptable salt thereof is present in the aqueous carrier at a concentration of at least 0.09M. In some embodiments, the Avizafone or a pharmaceutically acceptable salt thereof is present in the aqueous carrier at a concentration of at least 0.10M.

In some embodiments of the injectable pharmaceutical formulation, the formulation has a pH of between 5.5 and 8.0. In some embodiments, the pharmaceutically acceptable salt of Avizafone is a hydrochloride salt. In some embodiments, the pharmaceutically acceptable salt of Avizafone is derived from hydrobromic acid, sulfuric acid, nitric acid, phosphoric acid. In some embodiments, the pharmaceutically acceptable salt of Avizafone is a salt formed from one or more of the following: benzoic acid, benzenesulfonic acid, (1S)-(+)-10-camphorsulfonic acid, methanesulfonic acid, phosphoric acid, p-toluenesulfonic acid monohydrate, maleic acid, oxalic acid, fumaric acid, and malonic acid. In some embodiments, the aqueous carrier comprises less than 1.0 wt % of an organic solvent. In some embodiments, the aqueous carrier comprises less than 0.1 wt % of an organic solvent.

Some embodiments related to method of sedating a subject, comprising administering by injection to the subject an effective amount of any one of the injectable pharmaceutical formulations described herein.

Some embodiments related to method of sedating a COVID patient for intubation, comprising administering by injection to the COVID patient an effective amount of Avizafone for swift sedation prior to intubation.

In some embodiments, the injectable Avizafone may be used interchangeably with midazolam for treatment of a COVID patent.

Some embodiments related to a method of abating a seizure of a subject, comprising administering by injection to the subject an effective amount of any one of the injectable pharmaceutical formulations described herein.

In some embodiments, the injectable pharmaceutical formulation is administered to the subject by intramuscular injection. In some embodiments, the injectable pharmaceutical formulation is administered to the subject by subcutaneous injection. In some embodiments, the injectable pharmaceutical formulation is administered to the subject by intravenous injection.

In some embodiments, the effective amount of the injectable pharmaceutical formulation is an administration volume that is between 50 μL and 5.0 mL. In some embodiments, the administration volume is between 0.1 mL and 2.5 mL.

In some embodiments, the administering by injection does not produce significant pain at the injection site lasting more than 15 minutes beyond initial drug administration. The phrase “significant pain” refers to self-reported pain at a level of 2 or above on the 11-point Numeric Pain Rating Scale, wherein 0 represents “no pain” and 10 represents “worst pain imaginable.”

Some embodiments relate to administering an injectable pharmaceutical formulation described herein, wherein the peak maximum Diazepam concentration after administration occurs at least 25% faster than the peak maximum Diazepam concentration obtained by a comparable administration of an otherwise comparable injectable pharmaceutical formulation except that the Avizafone is present in the aqueous carrier of the otherwise comparable injectable pharmaceutical formulation at a concentration of 0.0176M. In some embodiments, the peak maximum Diazepam concentration after administration occurs at least 30% faster. In some embodiments, the peak maximum Diazepam concentration after administration occurs at least 35% faster. In some embodiments the peak maximum Diazepam concentration after administration occurs at least 40% faster.

### EXAMPLES

### Example 1: Evaluation of AVF, and Concentration-Time Pharmacokinetic Data Following IM or SQ Administration

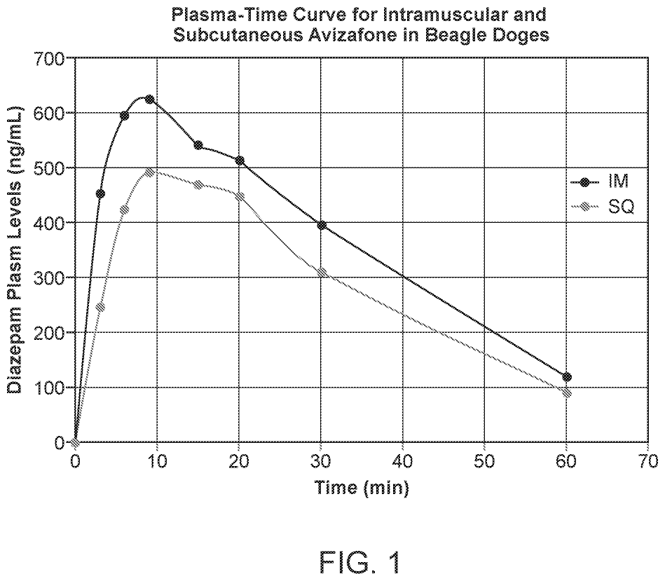

Experimental description: in this four-way non-randomized study, 12 beagle dogs were either dosed IM or SQ with the AVF formulations listed in Table 1. Water was the solvent for all administered doses. After administration, plasma samples were taken at various time points and the concentration of diazepam was determined (FIG. 1). Although niacin was given to half of the tested dogs, the presence of niacin had no statistical impact on the absorption rate of IM or SQ AVF delivery. In view of this, the data was pooled for each delivery method, yielding a net sample size of six dogs per method.

TABLE 1
AVF dosage information for dogs in Example 1.

| | | Solvent | AVF Dose | AVF Dose | Dog Mass | Dose (mg |
|---|---|---|---|---|---|---|
| | Dog | (mL)/Dose | (mg) | (mol/L) | (kg) | AVF/kg Dog) |
| IM | 1 | 1.31 | 18.4 | 0.0279 | 11.6 | 1.58 |
| | 2 | 1.24 | 17.4 | 0.0278 | 11.0 | 1.58 |
| | 3 | 1.47 | 20.6 | 0.0278 | 13.1 | 1.58 |
| | Mean | 1.34 | 18.8 | 0.0278 | 11.9 | 1.58 |
| IM + | 4 | 1.28 | 20.7 | 0.0321 | 11.3 | 1.83 |
| Niacin | 5 | 1.05 | 17.0 | 0.0321 | 9.3 | 1.83 |
| | 6 | 1.15 | 18.6 | 0.0321 | 10.2 | 1.82 |
| | Mean | 1.16 | 18.7 | 0.0321 | 10.3 | 1.83 |
| SQ | 7 | 0.49 | 16.6 | 0.0672 | 8.7 | 1.91 |
| | 8 | 0.45 | 15.2 | 0.0670 | 8.0 | 1.90 |
| | 9 | 0.56 | 18.9 | 0.0670 | 10.0 | 1.89 |
| | Mean | 0.50 | 16.9 | 0.0671 | 8.9 | 1.90 |
| SQ + | 10 | 0.60 | 19.3 | 0.0638 | 10.2 | 1.89 |
| Niacin | 11 | 0.60 | 19.3 | 0.0638 | 10.5 | 1.84 |
| | 12 | 0.60 | 19.3 | 0.0638 | 9.8 | 1.98 |
| | Mean | 0.60 | 19.3 | 0.0638 | 10.2 | 1.90 |

### Pharmacokinetic Data Analysis for Example 1

### Noncompartmental Analysis (NCA) of Mean Canine Data

The noncompartmental analysis (NCA) algorithm in Phoenix WinNonlin (WNL, version 6.4) assuming a one-compartment open model was used determine PK parameter estimates following single-dose intramuscular (IM) and subcutaneous (SQ) administration of 1.6 to 1.8 mg/kg avizafone without and with niacin to dogs. Mean (SD) PK parameters (i.e., Cmax, Tmax, AUClast, AUCinf, t1/2, V/F, CL/F, Cmax/D, AUClast/D, and AUCinf/D) were determined and confirmed by comparison to previously determined mean PK estimates.

### One-Compartment Open PK Model Fit of Data

The PK Model algorithm in Phoenix WNL (version 6.4, Model 3) assuming a one-compartment open model was used to provide the best fit of mean concentration-time determine routine PK parameter estimates following single-dose IM and SQ administration of 1.6 to 1.8 mg/kg avizafone without and with niacin to dogs.

The results of the PK analysis after single-dose administration of AVF via IM or SQ are summarized in Table 2 and Table 3, respectively.

TABLE 2
Mean (SD) Diazepam PK Parameters Following Single-dose IM
Administration of Approximately 1.6 to 1.8 mg/kg Avizafone
Without and With Niacin to Dogs Determined by NCA (N = 6)

| | | | | | | | Cmax/D |
|---|---|---|---|---|---|---|---|
| | Cmax | Tmax | AUClast | t½ | CL/F | V/F | (ng/mL)/ |
| | (ng/mL) | (min) | (min*ng/mL) | (min) | (mL/min/kg) | (mL/kg) | (mg/kg) |
| Mean | 669 | 17.5 | 21624 | 15.6 | 63.2 | 1435 | 397 |
| SD | 286 | (9-30) | 3826 | 1.78 | 7.37 | 316 | 182 |
| CV % | 42.8 | | 17.7 | 11.4 | 11.7 | 22.0 | 45.8 |

Tmax values are median and range.

TABLE 3
Mean (SD) Diazepam PK Parameters Following Single-dose SQ Administration of Approximately
1.9 mg/kg Avizafone Without and With Niacin to Dogs Determined by NCA (N = 6)

| | | | | | | | | Cmax/D |
|---|---|---|---|---|---|---|---|---|
| | Cmax | Tmax | AUClast | AUCinf | t½ | CUF | V/F | (ng/mL)/ |
| | (ng/mL) | (min) | (min*ng/mL) | (min*ng/mL) | (min) | (mL/min/kg) | (mL/kg) | (mg/kg) |
| Mean | 502 | 9 | 17039 | 19097 | 16.7 | 101 | 2432 | 264 |
| SD | 45.5 | (9-20) | 1724 | 2219 | 0.797 | 11.5 | 293 | 25.1 |
| CV % | 9.1 | | 10.1 | 11.6 | 4.8 | 11.4 | 12.0 | 9.5 |

Tmax values are median and range.

DZP absorption appeared to be most rapid following IM administration without niacin. Co-administration with niacin did not appear to influence the rate or extent of absorption of DZP favorably for IM or SQ administration.

Comparison of pooled DZP Cmax and AUC without and with niacin were higher (1.50-fold for Cmax/D and 1.43-fold for AUClast/D) following IM administration than following SQ administration. The data shows that although the total area under the curve is less for SQ administration, indicating less absorption, the time to peak plasma concentration (tmax) is nearly identical for both SQ and IM. Correcting for the fraction absorbed, the SQ dose provides a rapid onset AVF dose applicable and beneficial in both human and veterinary clinical settings. Additionally, although the total amount of diazepam absorbed in the first 60 minutes favored the IM route, the time to peak maximum concentration favored SQ. Therefore, the data shows that high concentration formulations of AVF can be administered with a low delivery volume for both IM and SQ routes of delivery. When the dose for SQ is increased to accommodate the lower total amount absorbed, then the onset and duration of sedation is similar for both dosage forms. This has not been shown previously for benzodiazepines and opens a new route of diazepam delivery for rescue formulations without using co-solvents. Based on the unexpected result that the SQ route led to a faster Tmax, it was hypothesized that the IM rate and consistency could be improved by using a solution that was more concentrated and thus lower in delivery volume.

### Example 2: Effect of Concentration and Volume on the Rate of Sedation in a Canine Model

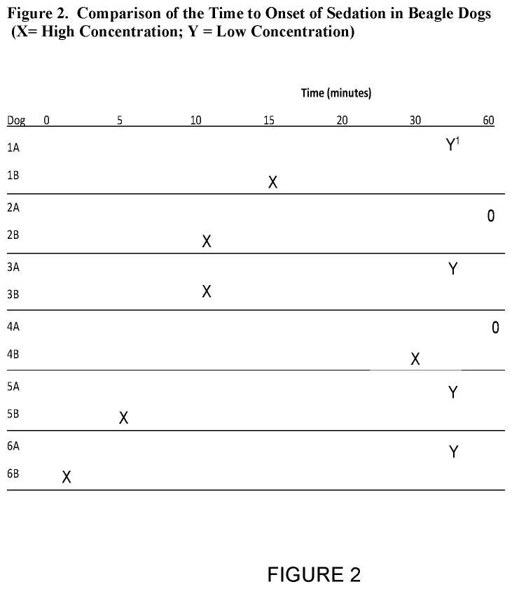

Experimental description: the six beagle dogs that were given IM doses of AVF in Example 1 were subsequently given more concentrated doses of AVF. The Example 1 data was collected in Study 1 during Month 1, and the more concentrated doses were given in Study 2 during Month 3. The AVF formulations administered are listed in Table 4. After administration, the time to onset of sedation was measured. These results are summarized in FIG. 2. The data from the Study 1 experiments is listed in the rows marked with the letter “A” and the date from the Study 2 experiments is listed in the rows marked with the letter “B.” An “X” is used to mark the approximate time point at which sedation began for Study 2 and a “Y” indicates onset of sedation for Study 1. If the onset of sedation did not take place within 60 minutes from the administration of AVF, that entry is marked with a “0.” The median time for onset of sedation for the dogs in Study 1 (using less concentrated AVF formulations) was greater than 45 minutes, whereas it was approximately 10 minutes in Study 2 (using a more concentrated formulation).

TABLE 4
AVF dosage information for dogs in Example 2.

| | Study 1 | | | Study 2 | | |
|---|---|---|---|---|---|---|
| | AVF | | AVF | AVF | | |
| | Dose | Solvent | Dose | Dose | Solvent | AVF Dose |
| Dog | (mg) | (mL)/Dose | (mol/L) | (mg) | (mL)/Dose | (mol/L) |
| 1 | 18.4 | 1.31 | 0.0279 | 19.90 | 0.40 | 0.0952 |
| 2 | 17.4 | 1.24 | 0.0278 | 18.70 | 0.40 | 0.0952 |
| 3 | 20.6 | 1.47 | 0.0278 | 23.90 | 0.50 | 0.0952 |
| 4 | 20.7 | 1.28 | 0.0321 | 19.80 | 0.40 | 0.0952 |
| 5 | 17.0 | 1.05 | 0.0321 | 18.00 | 0.40 | 0.0952 |
| 6 | 18.6 | 1.15 | 0.0321 | 20.00 | 0.40 | 0.0952 |
| Mean | 18.8 | 1.25 | 0.0300 | 20.05 | 0.417 | 0.0952 |
| S.D. | 1.576 | | | 2.046 | | |

### Pharmacokinetic Data Analysis for Example 2

### One-Compartment Open PK Model Fit of Data

As above, the PK Model algorithm in Phoenix WNL (version 6.4, Model 3) assuming a one-compartment open model was used to provide the best fit of mean concentration-time determine routine PK parameter estimates following single-dose IM administration of AVF.

### NCA of Individual Diazepam (DZP) Concentration-time Data in Dogs

As above, diazepam (DZP) PK parameters determined by NCA following single-dose intramuscular (IM) administration of AVF to dogs in Study 2 are summarized in Table 5. DZP PK parameters following IM AVF administration from Study 1 are also presented.

TABLE 5
Summary Diazepam PK Parameters Determined by NCA Following Single-
dose IM Administration of AVF to Dogs in Study 2 and Following
Single-dose IM Administration of AVF to Dogs in Study 1

| | | | | | Cmax/D | AUClast/D |
|---|---|---|---|---|---|---|
| | Cmax | Tmax | AUClast | t½ | (ng/mL)/ | (min*ng/mL)/ |
| | (ng/mL) | (min) | (min*ng/mL) | (min) | (mg/kg) | (mg/kg) |
| N | 6 | 6 | 6 | 6 | 6 | 6 |
| Median | 675 | 6.0 | 24748 | 24.2 | 605 | 21903 |
| GeoMean | 726 | 5.5 | 24918 | 23.4 | 644 | 22101 |
| Mean | 745 | 6.5 | 24984 | 23.5 | 656 | 22176 |
| SD | 188 | 4.0 | 2010 | 2.40 | 139 | 2009 |
| CV % | 25.2% | 61.3% | 8.0% | 10.2% | 21.3% | 9.1% |
| Minimum | 523 | 3 | 22549 | 18.9 | 484 | 19625 |
| Maximum | 991 | 12 | 28357 | 25.4 | 854 | 25095 |
| N | 6 | 6 | 6 | 3 | 6 | 6 |
| Median | 534 | 18 | 20590 | 16.6 | 574 | 22225 |
| GeoMean | 624 | 15.6 | 21349 | 15.5 | 650 | 22243 |
| Mean | 669 | 17.2 | 21624 | 15.6 | 702 | 22637 |
| SD | 286 | 8.0 | 3826 | 1.78 | 322 | 4685 |
| CV % | 42.8% | 46.5% | 17.7% | 11.4% | 45.8% | 20.7% |
| Minimum | 439 | 9 | 16976 | 13.5 | 447 | 16420 |
| Maximum | 1130 | 30 | 27134 | 16.7 | 1266 | 30397 |

Tmax values are median and range. GeoMean is geometric mean.
AUClast for Study 1 is equal to AUC0-60, since Tlast was 60 minutes for all data sets.

NCA and compartment modeling analysis of individual DZP concentration-time data were performed on data from two studies. DZP Cmax and AUC estimates and dose-normalized Cmax and AUC estimates determined by NCA following IM administration in Study 2 with a 3-fold higher solution concentration (and therefore a 3-fold smaller injection volume) were generally similar to those observed following IM administrations in Study 1. However, differences in DZP absorption rate and variability in PK parameter estimates were observed.

Evaluation of the rate of DZP absorption was best described by comparison of Cmax, Tmax, and K01. Cmax occurred most rapidly following IM administration in Study 2 (median Tmax=6 minutes and 18 minutes, respectively). Based upon compartment modeling, mean K01 estimates were 4.2-fold higher for DZP following IM administration in Study 2 (0.95008 1/min) when compared to Study 1 (0.22890 1/min). These input rate constants corresponded to mean half-life estimates for the absorption phase of 1.61 minutes (Study 2) and 4.90 minutes (Study 1). In addition, variability of the dose normalized parameters of Cmax and AUC were noticeably lower in Study 2 compared to Study 1.

These findings indicate that the 3-fold higher injection solution concentration and 3-fold smaller injection volume used in Study 2 resulted in nearly identical DZP exposure (i.e., Cmax/Dose and AUC/Dose) and substantially more rapid absorption (i.e., 3-fold shorter Tmax). This more rapid absorption took place despite the fact that a comparable amount of AVF was administered. Thus, the higher concentration solutions may be administered in situations when rapid onset of sedation is a priority. Such a significant improvement in a rate of onset of action was unexpected and has not been shown previously for any of the typical CNS drugs which are used to treat seizures, agitation, and aggression in clinical settings.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The subject matter described herein may be embodied in other specific forms without departing from the spirit or essential attributes thereof and, accordingly, reference should be made to the appended claims, rather than to the foregoing specification, as indicating the Scope of the subject matter described herein.

What is claimed is:

1. A method of treating acute agitation and aggression when sedation of a subject is required, comprising:
administering by injection to the subject a therapeutically effective dose of an injectable pharmaceutical formulation comprising:
an aqueous carrier; and
a pharmaceutically acceptable salt of Avizafone;
wherein a median time to the maximum Diazepam plasma concentration following injection to a subject occurs at least 25% faster than a comparable dose of a commercially available Diazepam Injection, USP.

2. The method of claim 1, wherein the Avizafone is present in the aqueous carrier at a concentration between 0.03M and 0.4M.

3. The method of claim 1, wherein the Avizafone is present in the aqueous carrier at a concentration between 0.05M and 0.15M.

4. The method of claim 1, wherein the pharmaceutically acceptable salt of Avizafone is a salt formed from one or more inorganic or organic acids including but not limited to: HBr, HCl, benzoic acid, benzenesulfonic acid, (1S)-(+)-10-camphorsulfonic acid, methanesulfonic acid, phosphoric acid, p-toluenesulfonic acid monohydrate, maleic acid, oxalic acid, fumaric acid, and malonic acid.

5. The method of claim 1, wherein the aqueous carrier comprises less than 1.0 wt % of an organic solvent.

6. The method of claim 1, wherein the aqueous carrier comprises less than 0.1 wt % of an organic solvent.

7. The method of claim 1, wherein the injectable pharmaceutical formulation is administered to the subject by intramuscular injection.

8. The method of claim 1, wherein the injectable pharmaceutical formulation is administered to the subject by subcutaneous injection.

9. The method of claim 1, wherein the injectable pharmaceutical formulation is administered to the subject by intravenous injection.

10. The method of claim 1, wherein the effective amount of the injectable pharmaceutical formulation is administered in a volume that is between 50 μL and 5.0 mL.

11. The method of claim 1, wherein the administration volume is between 0.1 mL and 2.5 mL.

12. The method of claim 1, wherein the agitation and aggression as caused by alcohol withdrawal symptoms; a drug overdose, mental disturbance, metabolic disorder, dementia, or Alzheimer's disease.

13. The method of claim 1, wherein the administration by injection does not produce significant residual pain at the injection site.

14. The method of claim 1, wherein the administering by injection does not produce significant pain at the injection site lasting more than 5 minutes beyond initial drug administration in no more than 10% of subjects.

15. The method of claim 1, wherein the median time to peak maximum Diazepam plasma concentration after intramuscular administration of Avizafone occurs at least 25% faster than the median time to maximum Diazepam plasma concentration obtained by a comparable dose administered via a commercially available Diazepam Injection, USP

16. The method of claim 15, wherein the maximum Diazepam concentration after administration occurs at least 35% faster.

17. The method of claim 1, wherein the Avizafone aqueous formulation is mixed with an antipsychotic drug, and or an antihistamine for administration in the same syringe.

18. The method of claim 17, wherein the antipsychotic drugs include but are not limited to, Haloperidol, Droperidol, Olanzapine, Aripiprazole, Ziprasidone, and Loxapine.

19. The method of claim 17, wherein the antihistamine drugs include but are not limited to, Promethazine, Diphenhydramine, Hydroxyzine, and Dimenhydrinate.

20. A method of treating acute agitation and aggression when sedation of a subject is required, comprising:
administering by injection to the subject a therapeutically effective dose of an injectable pharmaceutical formulation in a volume between 50 μL and 5.0 mL comprising:
an aqueous carrier with less than 1.0 wt % of an organic solvent; and
a pharmaceutically acceptable salt of Avizafone at a concentration between 0.03M and 0.4M in the aqueous carrier;
wherein a median time to the maximum Diazepam plasma concentration following injection to a subject occurs at least 25% faster than a comparable dose of a commercially available Diazepam Injection, USP.
